(19) United States
(12) Patent Application Publication
(10) Pub. No.: US 2013/0158296 A1
(43) Pub. Date: Jun. 20, 2013

(54) PROCESS FOR THE SYNTHESIS OF 1-AMINO-3-HALO-4,6-DINITROBENZENE

(71) Applicants: Annalisa Hargis, Wilmington, DE (US); Joachim C. Ritter, Wilmington, DE (US)

(72) Inventors: Annalisa Hargis, Wilmington, DE (US); Joachim C. Ritter, Wilmington, DE (US)

(73) Assignee: E I DU PONT DE NEMOURS AND COMPANY, Wilmington, DE (US)

(21) Appl. No.: 13/517,052

(22) PCT Filed: Dec. 21, 2010
(86) PCT No.: PCT/US10/61465

§ 371 Date: Aug. 8, 2012

Related U.S. Application Data
(60) Provisional application No. 61/288,436, filed on Dec. 21, 2009.

(51) Int. Cl. C07C 209/10

(52) U.S. Cl. CPC C07C 209/10
USPC 564/406

(57) ABSTRACT

A process is provided for the preparation of 1-amino-3-halo-4,6-dinitrobenzene and related compounds by monoamination of dihalodinitrobenzenes by ammonia in the presence of solvent and water. Using glycol as a solvent for the dihalodinitrobenzene and feeding ammonia at a rate at which it is consumed allows for the synthesis of high purity product, such as 1-amino-3-chloro-4,6-dinitrobenzene, at high yields.

This application claims priority under 35 U.S.C. §119(e) from, and claims the benefit of, U.S. Provisional Application No. 61/288,436, filed Dec. 21, 2009, which is by this reference incorporated in its entirety as a part hereof for all purposes.

### TECHNICAL FIELD

This disclosure relates to a method of making 1-amino-3-halo-4,6-dinitrobenzene and related compounds.

### BACKGROUND

The compound 1-amino-3-halo-4,6-dinitrobenzene (“AHDNB”), which is represented by the structure of the following Formula (I):

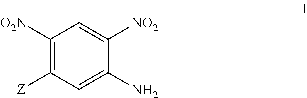

wherein Z is Cl or Br, can be used as a starting material or intermediate in the preparation of a variety of products, which include dyes, pesticides, and monomers for incorporation into polybenzimidazole polymers.

AHDNB can be made by oxidative amination using liquid ammonia and KMnO₄, according, for example, to the method described in Polish Patent No. 162,466, and in B. Szpakiewicz and M. Grzegożek, Russian Journal of Organic Chemistry, 40(6), (2004), 829-833 (translated from Zhurnal Organicheskoi Khimii, 40(6), (2004), 869-872). However, KMnO₄ in liquid ammonia is a hazardous combination, and the process produces quantitative amounts of Mn waste. McFarlane et al. [Journal of the Chemical Society, Perkin Transactions 1: Organic and Bio-Organic Chemistry (1972-1999), 3, (1988), 691-696] attempts the selective amination of dichlorodinitrobenzene using large amounts of aqueous ammonia (greater than twenty equivalents) in ethanol as a solvent which increases the cost of the process. Furthermore, the purity of the crude reaction product is less than desired, and recrystallization is needed to produce high purity product. Other reported syntheses are high-cost, multistep processes.

There thus remains a need for an improved process for making 1-amino-3-halo-4,6-dinitrobenzene and related compounds.

### SUMMARY

The inventions disclosed herein include processes for the preparation of 1-amino-3-halo-4,6-dinitrobenzene and related compounds.

Features of certain of the processes of this invention are described herein in the context of one or more specific embodiments that combine various such features together. The scope of the invention is not, however, limited by the description of only certain features within any specific embodiment, and the invention also includes (1) a subcombination of fewer than all of the features of any described embodiment, which subcombination may be characterized by the absence of the features omitted to form the subcombination; (2) each of the features, individually, included within the combination of any described embodiment; and (3) other combinations of features formed by grouping only selected features taken from two or more described embodiments, optionally together with other features as disclosed elsewhere herein. Some of the specific embodiments of the processes hereof are as follows:

In one embodiment, a process is provided for preparing a compound represented by Formula (II):

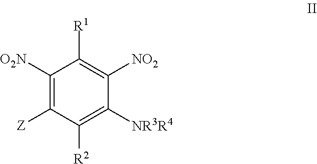

wherein Z is Cl or Br; R¹ and R² are each independently H, alkyl, or aryl; and R³ and R⁴, are each independently alkyl or aryl or may be joined to form an aliphatic ring structure;
   by forming a reaction mixture comprising a compound represented by the structure of the following Formula (III)

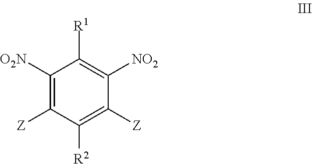

and solvent, in the presence of ammonia and about 2 to about 25 wt % water, wherein the ammonia is fed at about the rate at which it is consumed; and heating the reaction mixture between about 60° C. and about 140° C. to convert the compound represented by the structure of Formula (III) to the compound represented by the structure of Formula (II).

An advantageous effect of a process hereof is that adding ammonia at about the rate at which it is consumed at a temperature between about 60° C. and about 140° C. allows for the synthesis of high purity Formula (II) compounds in excellent space time yields.

### DETAILED DESCRIPTION

In one embodiment of a process hereof, a process is provided for preparing the compound represented by Formula (II):

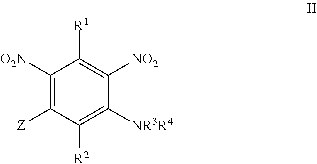

wherein Z is Cl or Br; R¹ and R² are each independently H, alkyl, or aryl; and R³ and R⁴, are each independently alkyl or aryl or may be joined to form an aliphatic ring structure;
   by forming a reaction mixture comprising a compound represented by Formula (III)

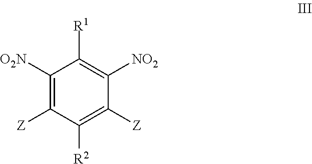

and solvent, in the presence of ammonia and about 2 to about 25 wt % water, wherein the ammonia is fed at about the rate at which it is consumed; and heating the reaction mixture between about 60° C. and about 140° C. to convert the compound represented by Formula (III) to the compound represented by Formula (II).

As used herein, the term “alkyl” denotes (a) a C₁˜C₁₂, or C₁˜C₈, C₁˜C₆, or C₁˜C₄, straight-chain or branched, saturated or unsaturated, substituted or unsubstituted, hydrocarbyl radical; or (b) a C₃˜C₁₂, or C₃˜C₆, cyclic aliphatic, saturated or unsaturated, substituted or unsubstituted, hydrocarbyl radical that is either bonded directly to the ring or to N or O, or is bonded to the ring or to N or O through a C₁˜C₆ straight-chain or branched, saturated or unsaturated, substituted or unsubstituted, hydrocarbyl radical. A C₁˜C₁₂ straight-chain or branched, saturated or unsaturated, substituted or unsubstituted, hydrocarbyl radical suitable for use herein may include, for example, a methyl, ethyl, n-propyl, i-propyl, n-butyl, sec-butyl, tent-butyl, n-pentyl, n-hexyl, n-octyl, trimethylpentyl, allyl and propargyl radical. An unsaturated aliphatic radical may include one or more double bonds, such as in a dienyl or terpenyl structure, or a triple bond such as in an acetylenyl structure. A C₃˜C₁₂ cyclic aliphatic, saturated or unsaturated, substituted or unsubstituted, hydrocarbyl radical suitable for use herein may include, for example, an alicyclic functional group containing in its structure, as a skeleton, cyclohexane, cyclooctane, norbornane, norbornene, perhydro-anthracene, adamantane, or tricyclo-[5.2.1.0^{2.6}]-decane groups.

As used herein, the term “aryl” denotes a C₆˜C₁₂, or C₆˜C₁₀, aromatic substituted or unsubstituted hydrocarbyl radical that is either bonded directly to the ring or to N or O, or is bonded to the ring or to N or O through a C₁˜C₆ straight-chain or branched, saturated or unsaturated, substituted or unsubstituted, hydrocarbyl radical. A C₆˜C₁₂ aromatic substituted or unsubstituted hydrocarbyl radical suitable for use herein may include, for example, a radical derived from a benzyl, phenyl, biphenyl, naphthyl, anthracenyl, xylyl, toluoyl or cumenyl structure; including, for example, a phenyl, methylphenyl, ethylphenyl, n-propylphenyl, n-butylphenyl, t-butylphenyl, p-chlorophenyl, p-bromophenyl, naphthyl or ethyl naphthyl radical.

An unsubstituted hydrocarbyl radical contains no atoms other than carbon and hydrogen. A substituted hydrocarbyl radical, however, is a radical in which
one or more heteroatoms selected from O, N, S and P may optionally be substituted for any one or more of the in-chain (i.e. non-terminal) or in-ring carbon atoms, provided that each heteroatom is separated from the next closest heteroatom by at least one and preferably two carbon atoms, and that no carbon atom is bonded to more than one heteroatom; and/or
one or more halogen atoms may optionally be bonded to a terminal carbon atom.
   In addition, however, a substituted C₃˜C₁₂ cyclic aliphatic, saturated or unsaturated hydrocarbyl radical, or a substituted C₆˜C₁₂ aromatic hydrocarbyl radical, may contain one or more C₁˜C₈, or C₁˜C₄, straight-chain or branched, saturated or unsaturated, hydrocarbyl radicals bonded to a carbon atom in the ring structure, such radical itself optionally being substituted with one or more heteroatoms selected from O, N, S and P, and/or containing one or more halogen atoms, subject to the conditions set forth above.

In one embodiment, R¹═R²═H, so that the compound represented by Formula (III) is 1,3-dihalo-4,6-dinitrobenzene (“DHDNB”) and the compound represented by Formula (II) is 1-amino-3-halo-4,6-dinitrobenzene (“AHDNB”). More specifically, when R¹═R²═H and each Z═Cl, the compound represented by Formula (III) is 1,3-dichloro-4,6-dinitrobenzene (“DCDNB”) and the compound represented by Formula (II) is 1-amino-3-chloro-4,6-dinitrobenzene (“ACDNB”).

In one embodiment of the process, the reaction mixture is formed by providing a suspension of the compound represented by Formula (III) in a mixture of solvent and water, then contacting the suspension with gaseous ammonia. The suspension contains about of the compound represented by Formula (III) in a mixture of solvent and about 2 to about 25 wt % water; and the suspension is heated to a temperature in the range of about 60° C. to about 140° C. and contacted with gaseous NH₃, for a time sufficient to convert the compound represented by Formula (III) to the compound represented by Formula (II).

More specifically in the above embodiment, the suspension and/or reaction mixture is heated to a temperature in the range of about 60° C. to about 140° C., preferably about 125° C. to about 135° C., and more preferably about 130° C., dissolving the 1,3-dihalo-4,6-dinitrobenzene in the solvent. The resulting solution is contacted at that temperature with gaseous ammonia for a time sufficient to convert the compound represented by Formula (III) to the compound represented by Formula (II), typically approximately two to four hours, close to ambient pressure; the gaseous ammonia is fed as it is consumed.

In another embodiment of the process, the reaction mixture is formed by forming a suspension of the compound represented by Formula (III) in solvent, and then contacting the suspension with an aqueous ammonia solution. More specifically, in this embodiment the reaction mixture is heated at a temperature in the range of about 60° C. to about 140° C., preferably about 100° C. to about 135° C., and more preferably about 130° C., dissolving the compound represented by Formula (III) in the solvent. The resulting solution is contacted at the selected temperature with aqueous NH₃ for a time sufficient to convert the compound represented by Formula (III) to the compound represented by Formula (II), typically for approximately two to four hours, close to ambient pressure. The aqueous NH₃ is fed as it is consumed, as indicated by analytical techniques such as pH measurements in the reaction solution or measuring NH₃ in the gas phase above the reaction solution. An advantage to this embodiment is that one can use an aqueous solution of ammonia (also referred to as ammonium hydroxide or “NH₄OH”) which is easier to handle and less hazardous than gaseous ammonia. Reaction rates are also higher when an aqueous solution of ammonia is used.

In yet another embodiment of the process hereof, rather than forming a suspension of a compound represented by Formula (III) in solvent and water and then feeding gaseous ammonia, or rather than forming a suspension of a compound represented by Formula (III) in solvent and then feeding an aqueous solution of ammonia, the compound represented by Formula (III) is instead contacted with a feed stream containing solvent, water and NH₃, thereby forming the reaction mixture. This allows for easy adjustment of the relative proportions of solvent, water and NH₃ at any time during the amination process.

This embodiment of the process thus involves contacting compound represented by Formula (III) with a feed that comprises solvent, water and NH₃ to form a reaction mixture that comprises a suspension of about 10 to about 25 wt % 1,3-dihalo-4,6-dinitrobenzene (based on the total weight of the whole reaction mixture) and about 2 to about 25 wt % water (based on the combined weight of water and solvent in the reaction mixture); and heating the reaction mixture to convert the compound represented by Formula (III) to a compound represented by Formula (III).

The total amount of water added should not significantly exceed the amount as compared to feeding 28% aqueous NH₃. That is, the water to NH₃ ratio should not exceed a ratio of about 5 to 1. The water added may be added at once prior to adding NH₃, but preferably it is added at the same rate and ratio as it would be added if 28% aqueous NH₃ was fed. The amount of water added may be between about 1 and about 5 times the amount of NH₃. Under those conditions, the product continuously precipitates as it is formed, reducing the formation of by-products. It is further preferred that the ammonia is added at the rate it is consumed. At low ammonia concentrations the solubility of the product is reduced; hence the rate of formation of by-products is reduced.

In any of the above embodiments, at reaction completion, the compound represented by Formula (III) thereby produced may be filtered, typically at about 60° C., and washed with a solvent such as glycol and then water. The mother liquor (filtrate) containing the solvent can be collected and the solvent distilled and recycled to the reaction mixture; when this is done, purges are drawn to prevent accumulation. A wet cake of the compound represented by Formula (II) can be dried if it is the final product. Alternatively, it can be slurried with water, or any other solvent suitable for use in subsequent processes, as a suspension and transferred to another reactor for further processing.

In any of the embodiments hereof, a solvent suitable for use includes an organic solvent inert to the reaction such as an aliphatic dihydric alcohol such as ethylene glycol (“glycol”). Using glycol as a solvent and adding aqueous ammonia at a rate as it is consumed at temperatures of 6° C.-140° C. allows for the high yield synthesis of high purity compounds represented by Formula (II), in particular, 1-amino-3-chloro-4,6-dinitrobenzene, in excellent space time yields. The product is directly isolated from the reaction mixture since it is only sparingly soluble in suitable solvents such as glycol at temperatures below 50° C. All impurities remain in solution.

DHDNB suitable for use herein may be prepared, for example, by nitration of 1,3-dihalobenzene as described in Knobloch et. al., Chem. Her. 91, 2563 (1958); or according to the method described in U.S. application Ser. No. 12/335,959, which is by this reference incorporated in its entirety as a part hereof for all purposes. Typically, the DHDNB used is 1,3-dichloro-4,6-dinitrobenzene (“DCDNB”); that is, each Z═Cl.

U.S. application Ser. No. 12/335,959 provides a process for preparing a 1,3-dihalo-4,6-dinitrobenzene by (a) admixing a 1,3-dihalobenzene, which is represented by the structure of the following Formula (IX):

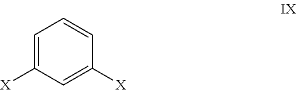

wherein each X is independently Cl or Br, with fuming nitric acid, sulfuric acid, and SO₃; to form a reaction mixture that is characterized by (i) a concentration of nitric acid therein that is in the range of about 2.0 to about 2.3 moles per mole of 1,3-dihalobenzene; (ii) a concentration of SO₃ therein that is in the range of about 1 to about 3 moles per mole of 1,3-dihalobenzene; (iii) a concentration of 1,3-dihalobenzene therein that is in the range of about 12 to about 24 weight percent; and (iv) a temperature of up to about 120° C.; and (b) stirring the reaction mixture at a temperature in the range of about −10° C. to about 70° C. to form a 1,3-dihalo-4,6-dinitrobenzene product. A 1,3-dihalo-4,6-dinitrobenzene product may be isolated from the reaction mixture at a temperature between about 0° C. and about 40° C.

In a preferred embodiment, any of the above steps of a process hereof can be run in the exclusion or substantial exclusion of oxygen, which can be accomplished by running under a blanket of nitrogen.

### EXAMPLES

The advantageous attributes and effects of the processes hereof may be seen in a series of examples as described below. The embodiments of these processes on which the examples are based are representative only, and the selection of those embodiments to illustrate the invention does not indicate that materials, reactants, conditions, steps, techniques, or protocols not described in these examples are not suitable for practicing these processes, or that subject matter not described in these examples is excluded from the scope of the appended claims and equivalents thereof.

In the examples, the meaning of certain abbreviations is as follows: “ACME” means 1-amino-3-chloro-4,6-dinitrobenzene, “DCDNB” means 1,3-dichloro-4,6-dinitrobenzene, “g” means gram(s), “GC” means gas chromatography, “h” means hour(s), “LC” means liquid chromatography, “mL” means milliliter(s), and “min” means minute(s).

As used herein, the term “net yield” of a product denotes the actual, in-hand yield, i.e. the theoretical maximum yield minus losses incurred in the course of activities such as isolating, handling, drying, and the like. As used herein, the term “purity” denotes what percentage of an in-hand, isolated sample is actually the specified substance.

### Example 1

### Synthesis of 1-amino-3-chloro-4,6-dinitrobenzene (ACDNB)

This example demonstrates that adding aqueous ammonia to an ethylene glycol suspension of 1,3-dichloro-4,6-dinitrobenzene leads directly to clean formation of ACDNB product in good yields

28 g of 1,3-dichloro-4,6-dinitrobenzene (0.114 mole) and 170 g of ethylene glycol, a 14% loading, were added to a 3-necked round bottom flask. A dropping funnel was loaded with 0.312 mole of ammonium hydroxide solution. The addition was started when the solution had reached an internal temperature of 130° C. at a rate such that the ammonia was consumed quantitatively (˜2-3 h) and no ammonia gas flow leaving the reactor was observed. The reaction mixture was cooled to 60° C., and the crystalline ACDNB product then filtered on a glass filter and washed with a little glycol, followed by water. The material was dried and 22 g product was collected for a net yield of 85%. An approximate additional 3 g remained in solution and can be recycled. The total reaction selectivity was greater than 95%. The collected ACDNB product was analyzed by GC and LC and its purity determined to be >98%.

### Example 2

### Synthesis of 1-amino-3-chloro-4,6-dinitrobenzene (ACDNB): kilogram scale

This example demonstrates the synthesis of ACDNB on a kg scale in excellent time space yields and high purity.

This synthesis of ACDNB was carried out as described in Example 1 but at a larger scale: 1066 g of 1,3-dichloro-4,6-dinitrobenzene (4.5 moles, 99% purity) in 5815 g of ethylene glycol were reacted with 553 g of 28% aqueous NH₃ (4.5 moles). The ammonia was added at the rate at which it was consumed over a period of 1.5 h. The crude reaction product was isolated by filtration and washed with 750 mL ethylene glycol followed by 750 mL of water and 400 mL of methanol. After drying, 821 g of ACDNB were isolated (85% yield). The purity was determined >96% by LC and GC.

The mother liquor was analyzed and it was found that an approximate 90 g of product remained in solution and may be recycled.

### Comparative Example A

### Preparation of ACDNB from DCDNB Using Aqueous Ammonia in Ethanol

This example demonstrates that adding aqueous ammonia to an ethanol suspension of 1,3-dichloro-4,6-dinitrobenzene does not lead to a clean reaction product.

A three-necked flask was equipped with a thermometer, magnetic stirrer, and reflux condenser with a nitrogen bubbler. The ammonium hydroxide (200 mL, 28% solution) was added to the DCDNB (0.13 moles) in 300 mL of ethanol, and the mixture heated under reflux (80° C.) for 3 h. The reaction was monitored by LC analysis until all 1,3-dichloro-4,6-dinitrobenzene starting material was consumed. Upon reaction completion the red solution with solids was allowed to cool to room temperature before it was filtered. The yellow-to-bronze colored fine crystalline product was washed with water, then cold ethanol. The net yield was 22 g and the purity by LC was 80% with 20% 1,3-diamino-4,6-dinitrobenzene impurity.

### Comparative Example B

### Preparation of ACDNB from DCDNB in Glycol Using Excess Aqueous Ammonia

This example shows that adding excess aqueous ammonia to an ethylene glycol suspension of 1,3-dichloro-4,6-dinitrobenzene does lead to a cleaner reaction product.

A three-necked flask was equipped with a thermometer, magnetic stirrer, and reflux condenser with a nitrogen bubbler. The ammonium hydroxide (200 mL, 28% solution) was added to 1,3-dichloro-4,6-dinitrobenzene (“DCDNB”) (0.13 moles) in 300 mL of ethylene glycol, and the mixture heated to 80° C. for about 3 h. The reaction was monitored by LC analysis until all 1,3-dichloro-4,6-dinitrobenzene starting material was consumed. Upon reaction completion the red solution with solids was allowed to cool to room temperature before it was filtered. The yellow-to-bronze colored fine crystalline product was washed with water, then cold ethanol. The net yield was 24 g and the purity was 91% with about 9% 1,3-diamino-4,6-dinitrobenzene impurity.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the invention as described herein. Where a range of numerical values is stated herein as being less than a stated value, the range is nevertheless bounded on its lower end by a non-zero value.

In this specification, unless explicitly stated otherwise or indicated to the contrary by the context of usage, where an embodiment of the subject matter hereof is stated or described as comprising, including, containing, having, being composed of or being constituted by or of certain features or elements, one or more features or elements in addition to those explicitly stated or described may be present in the embodiment. An alternative embodiment of the subject matter hereof, however, may be stated or described as consisting essentially of certain features or elements, in which embodiment features or elements that would materially alter the principle of operation or the distinguishing characteristics of the embodiment are not present therein. A further alternative embodiment of the subject matter hereof may be stated or described as consisting of certain features or elements, in which embodiment, or in insubstantial variations thereof, only the features or elements specifically stated or described are present.

In this specification, unless explicitly stated otherwise or indicated to the contrary by the context of usage, amounts, sizes, ranges, formulations, parameters, and other quantities and characteristics recited herein, particularly when modified by the term “about”, may but need not be exact, and may also be approximate and/or larger or smaller (as desired) than stated, reflecting tolerances, conversion factors, rounding off, measurement error and the like, as well as the inclusion within a stated value of those values outside it that have, within the context of this invention, functional and/or operable equivalence to the stated value.

What is claimed is:

1. A process for preparing a compound represented by Formula (II): 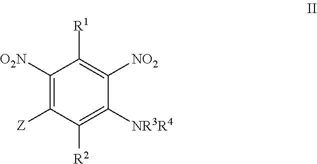
wherein Z is Cl or Br; R¹ and R² are each independently H, alkyl, or aryl; and R³ and R⁴, are each independently alkyl or aryl or may be joined to form an aliphatic ring structure;
by forming a reaction mixture comprising a compound represented by the structure of the following Formula (III) 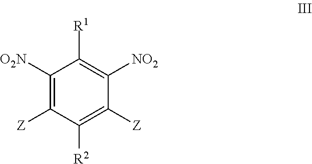
and solvent, in the presence of ammonia and about 2 to about 25 wt % water, wherein the ammonia is fed at about the rate at which it is consumed; and heating the reaction mixture between about 60° C. and about 140° C. to convert the compound represented by the structure of Formula (III) to the compound represented by the structure of Formula (II).

2. A process according to claim 1 wherein each Z is Cl.

3. A process according to claim 2 wherein Z═Cl and R¹, R², R³, and R⁴ are each H.

4. A process according to claim 1 wherein the solvent comprises an aliphatic dihydric alcohol.

5. A process according to claim 4 wherein the aliphatic dihydric alcohol is ethylene glycol.

6. A process according to claim 1 wherein the reaction mixture is prepared by contacting a suspension of a compound represented by Formula (III) in solvent and water with gaseous ammonia.

7. A process according to claim 1 wherein the reaction mixture is prepared by contacting a suspension of a compound represented by Formula (III) in solvent with an aqueous solution of ammonia.

8. A process according to claim 1 wherein the reaction mixture is prepared by contacting a compound represented by Formula (III) with a feed stream containing solvent, water and NH₃.

9. A process according to claim 8 wherein the amount of water is between about 1 and about 5 times the amount of NH₃.

10. A process according to claim 1 wherein the compound represented by Formula (II) thereby produced is filtered and washed with solvent and then water.

11. A process according to claim 10 wherein the filtrate containing solvent is collected and distilled, and the solvent is recovered and recycled to the reaction mixture.

12. A process according to claim 1 wherein the compound represented by Formula (II) thereby produced is slurried with water as a suspension and transferred to another reactor for further processing.

13. A process according to claim 1 further comprising:
(a) admixing a 1,3-dihalobenzene, which is represented by the structure of the following Formula (IX): 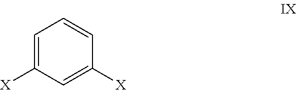
wherein each X is independently Cl or Br, with fuming nitric acid, sulfuric acid, and SO₃ to form a reaction mixture that is characterized by (i) a concentration of nitric acid therein that is in the range of about 2.0 to about 2.3 moles per mole of 1,3-dihalobenzene; (ii) a concentration of SO₃ therein that is in the range of about 1 to about 3 moles per mole of 1,3-dihalobenzene; (iii) a concentration of 1,3-dihalobenzene therein that is in the range of about 12 to about 24 weight percent; and (iv) a temperature of up to about 120° C.; and
(b) stirring the reaction mixture at a temperature in the range of about −10° C. to about 70° C. to form a 1,3-dihalo-4,6-dinitrobenzene product;
to provide a 1,3-dihalo-4,6-dinitrobenzene for incorporation into the reaction mixture in the process of claim 1.
